(19) United States
(12) Patent Application Publication
(10) Pub. No.: US 2026/0185979 A1
(43) Pub. Date: Jul. 2, 2026

(54) APPARATUSES AND METHODS FOR ANALYZING LIVE CELLS

(71) Applicant: AGILENT TECHNOLOGIES, INC., Santa Clara, CA (US)

(72) Inventors: Jyothi Thyagabhavan Mony, San Diego, CA (US); Yama Abassi, Temecula, CA (US); Brandon J. Lamarche, Santa Clara, CA (US)

(21) Appl. No.: 19/128,112

(22) PCT Filed: Nov. 7, 2023
(86) PCT No.: PCT/US2023/078963

§ 371 Date: May 7, 2025

Related U.S. Application Data
(60) Provisional application No. 63/383,245, filed on Nov. 10, 2022.
(60) Provisional application No. 63/580,658, filed on Sep. 5, 2023.

(51) Int. Cl. G01N 33/50; G01N 33/483

(52) U.S. Cl. CPC G01N 33/5011; G01N 33/4836

(57) ABSTRACT

Evaluating immune and cancer cells is provided for by assessing cytolysis of cancer cells by effector cells, which includes: providing a cell-substrate impedance monitoring device operably connected to an impedance analyzer, wherein the device comprises a well for receiving cells and an electrode array at a base of the well, the device further operably connected to an imaging unit; b) adding target cells characterized as cancer cells to the well; c) disposing a layer comprising an extracellular matrix (ECM) over the target cells; d) adding effector cells over the ECM layer; and e) imaging the well and monitoring cell-substrate impedance of the well to determine invasion of the effector cells through the ECM layer and effectiveness of effector cell killing of the target cells either directly or via migration and invasion through extracellular matrix.

### CROSS-REFERENCE TO RELATED APPLICATIONS

This application claims the benefit of U.S. Provisional Application No. 63/383,245, filed Nov. 10, 2022 and U.S. Provisional Application No. 63/580,658, filed Sep. 5, 2023. The contents of the aforementioned applications are hereby incorporated by reference in its entirety.

### BACKGROUND

The tumor microenvironment (TME) of solid tumors poses significant challenges to immune response and recapitulating the characteristics of TME in assays can be critical for identifying and refining cellular immunotherapies. The engineered cytotoxic natural killer (NKs) and cytotoxic T (CD8⁺T) cells have to extensively infiltrate the solid tumors to execute effector functions. During migration, the lymphocytes have to navigate through an acellular space with structural scaffolding constituting the extracellular matrix (ECM). The ECM can modulate a wide array of cellular responses in both resident tumor cells and infiltrating lymphocytes, thus determining the outcome. Thus, there is a need to develop methods and systems to evaluate both tumor cell and immune cell response in the context of an ECM.

### SUMMARY

In an aspect, the disclosure provides a method of assessing cytolysis of cancer cells by effector cells. The method comprises: a) providing a cell-substrate impedance monitoring device operably connected to an impedance analyzer, wherein the device comprises a well for receiving cells and an electrode array at a base of the well, the device further operably connected to an imaging unit; b) adding target cells characterized as cancer cells to the well; c) disposing a layer comprising an extracellular matrix (ECM) over the target cells; d) adding effector cells over the ECM layer; and e) imaging the well and monitoring cell-substrate impedance of the well to determine invasion of the effector cells through the ECM layer and effectiveness of effector cell killing of the target cells either directly or via migration and invasion through extracellular matrix.

In some embodiments, the imaging unit further comprises an imaging device disposed adjacent to the well.

In some embodiments, the cancer cell is from a solid tumor. In some embodiments, the effector cell is an immune cell. In some embodiments, the effector cell is a natural killer (NK) cell. In some embodiments, the effector cell is a T cell. In some embodiments, the T cell is a CD8⁺T cell. In some embodiments, the effector cell expresses a chimeric antigen receptor (CAR). In some embodiments, the NK cell is a chimeric antigen receptor (CAR) CAR-NK cell. In some embodiments, the T cell is a chimeric antigen receptor (CAR) CAR-T cell.

In some embodiments, the cancer cell has aberrant FGFR signaling. In some embodiments, the method further comprises adding an FGFR inhibitor to the well. In some embodiments, the FGFR inhibitor comprises pemigatinib.

In another aspect, the disclosure provides a system configured to perform a method of assessing cytolysis of cancer cells by effector cells as described herein.

In yet another aspect, the disclosure provides a method, comprising: measuring cell-substrate impedances between target cells and effector cells separated by a layer of an extracellular matrix (ECM) at various times during an assay; capturing images the effector cells and the target cells at various times during the assay; and determining an effectiveness of the effector cells relative to the target cells based on changes to the cell-substrate impedance the images over a time period of the assay.

In some embodiments, the method further comprises: applying a first dye of a first color to the target cells; and applying a second dye of a second color, different than the first color, to the effector cells.

In some embodiments, the method further comprises: varying a thickness of the ECM in multiple wells of a multiwell plate in which the target cells and effector cells are disposed.

In some embodiments, the method further comprises: applying a pharmaceutical compound of interest to one or more of the target cells, the ECM, and the effector cells; comparing a baseline effectiveness of the effectiveness the effector cells relative to the target cells without the pharmaceutical compound of interest applied versus an experimental effectiveness of the effectiveness the effector cells relative to the target cells with the pharmaceutical compound of interest applied; and in response to the experimental effectiveness satisfying an effectiveness threshold, using the pharmaceutical compound of interest for treatment or prophylaxis in a biological subject of a disorder associated with the target cells.

In some embodiments, the method further comprises: applying a first pharmaceutical compound of interest to a first subset of one or more of the target cells, the ECM, and the effector cells; applying a second pharmaceutical compound of interest to a second subset, separate from the first subset, of one or more of the target cells, the ECM, and the effector cells; comparing a first effectiveness of the effectiveness the effector cells relative to the target cells with the first pharmaceutical compound of interest applied versus a second effectiveness of the effectiveness the effector cells relative to the target cells with the second pharmaceutical compound of interest applied; and in response to the first pharmaceutical compound of interest having a greater effectiveness than the second pharmaceutical compound of interest, using the first pharmaceutical compound of interest for treatment or prophylaxis in a biological subject of a disorder associated with the target cells.

In still another aspect, the disclosure provides a method of treating a solid tumor, comprising: administering a therapeutically effective amount of an FGFR inhibitor and a chimeric antigen receptor (CAR) therapy to a subject in need thereof.

In some embodiments, the solid tumor has aberrant FGFR signaling. In some embodiments, the FGFR inhibitor is an inhibitor of one, two, three, or all of FGFR1, FGFR2, FGFR3, or FGFR4. In some embodiments, the FGFR inhibitor is an FGFR1 inhibitor. In some embodiments, the FGFR inhibitor is an FGFR2 inhibitor. In some embodiments, the FGFR inhibitor is an FGFR3 inhibitor. In some embodiments, the FGFR inhibitor is an FGFR4 inhibitor. In some embodiments, the FGFR inhibitor comprises pemigatinib.

In some embodiments, the FGFR inhibitor is administered prior to the CAR therapy is administered. In some embodiments, the FGFR inhibitor is administered concurrently with the administration of the CAR therapy. In some embodiments, the FGFR inhibitor is administered after administration of the CAR therapy.

In some embodiments, the CAR therapy is a CAR T cell (CAR-T) therapy, a CAR-NK cell therapy, a CAR-macrophage therapy, or a CAR-gd-T therapy.

### BRIEF DESCRIPTION OF THE DRAWINGS

FIGS. 1A-1B depict the delayed target cell killing with increased distance for invasion, according to embodiments of the present disclosure. Addition of NK92 at 24 hours (h) (E:T 3:1) results in a drop in cellular impedance (CI) due to the killing of target cells (FIG. 1A). Target cell loss from NK cytotoxicity is delayed in matrigel and slows down with increasing volumes representing increasing invasion distances. Live cell imaging confirms the delay in loss of target cells in the visual fields (FIG. 1B).

FIGS. 2A-2B depict representative images showing NK cytotoxicity in increasing volumes of matrigel, according to embodiments of the present disclosure. The eLive Green stains NK-92 cells (FIG. 2A) but not healthy Michigan Cancer Foundation-7 (MCF-7) cells (FIG. 2B). The progressive loss of MCF-7 (red) targets when NK-92 cells are added over matrigel layer is accompanied by increased green fluorescence suggesting an accumulation of NK-92 and dead target cells. Morphological changes are evident at earlier timepoints (˜45-55 h).

FIGS. 3A-3C depict that the delay target cell killing by NK cells with inhibition of MMPs, according to embodiments of the present disclosure. The drop in impedance (CI) from killing of target cells is delayed but not abrogated by GM6001 (FIG. 3A). Delayed loss in red fluorescence signals of MCF7 and increase in Green fluorescence (eLive) in the presence of the MMP inhibitor were observed (FIG. 3B). Percent cytolysis [(1−(treated/untreated)*100] calculated at 72 h shows slower rates of cytolysis with increased matrigel and matrix metalloproteinase (MMP) inhibitor (FIG. 3C).

FIG. 4 is a diagram showing an example ECM invasion and cytotoxicity assay, according to embodiments of the present disclosure.

FIGS. 5A-5G illustrate that Pemigatinib increases the effectiveness of EpCAM-CAR-T in squamous cell carcinoma, according to embodiments of the present disclosure. The expression of EpCAM on cell surface of different tumor cells (FIG. 5A). The normalized cell index over time shows that FGFR1 inhibitor reduces the proliferation of A431 cells (FIG. 5B), EpCAM CART target A431 cells in cytotoxicity assays (FIG. 5C), EpCAM CART (E:T ˜3.5) are ineffective against A431 cells growing in Matrigel (FIG. 5D), the CAR-T cells effectively control A431 cell proliferation in the presence of FGFRi (despite EpCAM CART (E:T ˜3.5) being ineffective against A431 cells in Matrigel) (FIG. 5E), and that Percent Cytolysis computed from impedance readings and live cell imaging data confirms increased efficacy of EpCAM CART in the presence of Pemigatinib (FIG. 5F), which are confirmed via representative images taken at the 100 h mark (FIG. 5G).

FIGS. 6A and 6B illustrate an experimental assay setup, according to embodiments of the present disclosure. The instrument, wellplate, and comparison of the setup in one of the wells with a Boyden chamber is shown (FIG. 6A). Advantages of this setup with respect to data collection of the classical approaches are outlined with the steps in the assay (FIG. 6B).

FIGS. 7A-7C depict that the cytotoxicity of NK-92 is delayed with increasing invasion distance, according to embodiments of the present disclosure. The addition of NK-92 at 24 hours (E:T=3:1) results in a drop in impedance (CI) due to cytolysis of targets (FIG. 7A), and the drop in impedance is delayed in matrigel (FIG. 7B). Cytolysis is further delayed in increasing volumes of matrigel (FIG. 7C). Percent cytolysis was calculated with reference to untreated MCF-7 growing in matrigel.

FIGS. 8A and 8B depict that MMP inhibition delays target cell killing by NK cells, according to embodiments of the present disclosure. FIG. 8A shows that cytolysis is reduced GFP-NK92 at 24 h (E:T=3: 1) results in a drop in impedance (CI) due to the killing of target cells. FIG. 8B shows that live cell imaging confirms the reduced loss of target cells (red fluorescence) in the visual fields in the presence of MMP inhibitor (2 μM and 10 μM).

FIG. 9 depicts representative images for clustering of MCF-7 and progressive loss red fluorescence associated with cytotoxicity. A few green GFP-NK92 cells (yellow outline as indicated in FIG. 9) make multiple contacts with MCF7-red target cells in the clusters resulting in cell death over the course of the assay.

### DETAILED DESCRIPTION

The present disclosure relates, at least in part, to a co-culture model for real time assessment of immune cell invasion and killing of tumor cells.

Extracellular matrix (ECM) is a generic term for the diverse types of acellular structural component in tissues that play an important role in homeostasis. Without wishing to be bound by theory, it is believed that in some embodiments, the ECM is an intricate network composed of an array of multidomain macromolecules organized in a cell/tissue-specific manner. For example, major ECM components may include collagens, proteoglycans, elastin, and/or cell-binding glycoproteins, each with distinct physical and biochemical properties. The ECM in solid tumors is greatly altered and can contribute to the modulation of immune cell function in the tumor microenvironment. Lymphocytes such as the cytotoxic natural killer (NK) and cytotoxic T (CD8⁺T) cells that are harnessed in immunotherapy can be regulated by the ECM. As detailed herein, the matrigel layer presents a challenge for the NK cells and increasing the distance for invasion delays the kinetics of tumor cell killing. Furthermore, invasion and killing of tumor targets depends on the ability of the NK or CD8⁺T cells to remodel the matrix with matrix-metalloproteinases.

To assess the effectiveness of the cells in attacking targets in ECM, an experimental or assay setup with increasing volumes of matrigel (used to represent increasing distance for invasion) are layered over target tumor cells expressing a fluorescent protein. Immune cells are seeded over the solidified matrigel layer, and a second marker of a different color than the fluorescent protein is added to the wells. The invasion and function of the immune cells evaluated using impedance-based measurements of immune cell killing and live cell imaging data collected on the XCELLIGENCE RTCA ESIGHT® imaging and sensing system, available from Agilent Tech., Inc. of Santa Clara, California, USA).

Impedance increases with time as MCF-7 target cells adhere and proliferate; however, the addition of NK-92 cells results in a drop in impedance due to killing of the target cells. The total time taken for impedance levels to fall to levels at the time of NK-92 addition increased progressively from 46 h for 50 mL/well to 72 h for 110 mL/well. The eLive green stained NK cells and dead cells, but not healthy MCF-7 targets. Consistent with impedance data, the loss of red fluorescence from target cells and increase in green fluorescence was progressively delayed with increasing volume of matrigel. A similar delay in kinetics was achieved with the broad spectrum MMP inhibitor GM6001 (e.g., 2 millimoles (mM) and 10 mM), suggesting that MMPs plays a role in the NK function. Interestingly, the NK-92 cells induce significant morphological changes in MCF-7-red target cells prior to invading all the way through the Matrigel, suggesting an early distal effect.

The results suggest a role for effector functions of NK cells potentially involving cytokines, that is independent from invasion and/or ability to degrade the matrix, for the killing of susceptible target cells. The assay also demonstrates the potential for adapting the XCELLIGENCE RTCA ESIGHT® imaging and sensing system to study various ECM interactions with immune cells.

Immune cells invade the extracellular matrix (ECM) to perform effector functions that include the killing of target cells. Traditionally, invasion and cytotoxicity functions are evaluated in separate end-point assays. A potential drawback is that cytotoxicity is not readily evaluated in the presence of ECM, which can modulate cellular responses of lymphocytes. Furthermore, the traditional assay systems can be cumbersome and often involves multiple steps. In some embodiments, the disclosure provides an easy to set up assay (e.g., in a 96 well format on the XCELLIGENCE RTCA ESIGHT® imaging and sensing system that combines impedance and imaging capabilities to deliver real-time readout of invasion and cytotoxicity functions. The methods and systems described herein are relevant for cancer immunotherapy. In some embodiments, the disclosure provides a real-time assay for simultaneously evaluating both invasion and cytotoxicity of immune cells. In some embodiments, the assay is used to systematically study the effect of ECM on immune cell-tumor cell interactions. In some embodiments, the methods and systems described herein avoid the drawbacks of complicated setups, such as use of microfluidics arrangements. Other benefits will be apparent to those of skill in art upon a detailed reading of the present disclosure.

The methods and systems described herein can have the capabilities of in evaluating both invasion and killing of target tumor cells by the immune cells (e.g., T cells, NKs, macrophages) in the context of an ECM (e.g., an ECM of any type of solid tumor described herein). Traditionally, invasion/migration and cytotoxicity functions are evaluated in separate assays. In some embodiments, the methods and systems described herein can collect impedance-based readouts of cytotoxicity and live cell imaging data simultaneously in real time, for example, in a 96 well format.

NKs are innate lymphocytes that eliminate infected, stressed, or transformed cells by identifying and distinguishing the “altered” cells from healthy cells using an array of activating and inhibiting receptors. NKs and engineered variants thereof have emerged as an attractive option alongside chimeric antigen receptor (CAR) T cells for adoptive immunotherapy.

The reasons for lack of efficacy of adoptive cell therapies against solid tumors include, for example, limited tumor specific antigens, immunosuppressive environment of the tumor itself leading to immune cell exhaustion, the physical infiltration of tumor stroma by the immune cells, and combinations thereof. While most in vitro assays for assessment of adoptive immune cells have focused on activation, proliferation and cytotoxicity, very few assays focus on the migration and extravasation of immune cell to the site of tumor. In some embodiments, the disclosure herein provides a real time in vitro assay that models both the migration/invasion of immune cells to the site of tumor, as well as the propensity of the immune cells to kill the tumor cells.

Recapitulating the tumor microenvironment in solid tumors could play a pivotal part in formulating and refining strategies as cellular immunotherapy for multiple cancers. ECM can be dysregulated in TME and ECM has the ability to modify the cellular responses. Thus, there is a need to evaluate both tumor cell and immune cell response in the context of an ECM. In this regard, various constituents of ECM can regulate the function of NK cells.

The ability of NKs to invade through the extracellular space to reach the target cells could depend on the ability to degrade ECM. NKs can express multiple MMPs, and NK-92 invasion is reduced in the presence of MMP inhibitor GM6001 in transmigration assays, which suggests a role for MMPs in the migration of NK cells through matrigel. Traditional invasion/migration potential and cytotoxicity functions are either evaluated separately in workflows or evaluated using complex setups.

### ECM Invasion and Cytotoxicity Assay

In some embodiments, NK invasion and function is evaluated using impedance-based measurements of immune cell killing (normalized cell index, CI) and live cell imaging on the XCELLIGENCE RTCA ESIGHT® imaging and sensing system (available from Agilent Tech., Inc.).

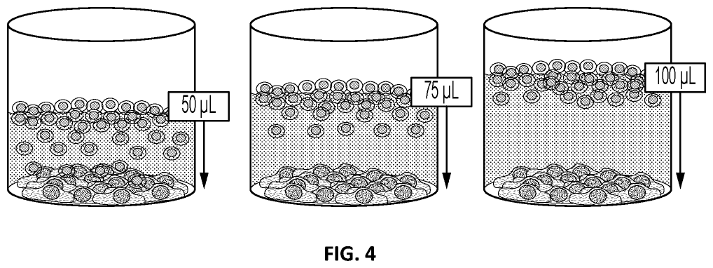

An exemplary protocol is described as follows. Briefly, varying volumes of matrigel (50-110 microliters (μL)/well; 6 milligrams (mg)/millilter (mL)) representing increasing distance for invasion is layered over MCF-7 (available from American Type Culture Collection (ATCC)) human breast adenocarcinoma cells stably expressing nuclear-localized red fluorescent protein (MCF-7). NK-92 cells (available from Creative Bioarray, ATCC) are added over the solidified matrigel layer at E:T of 3:1. eLive Green (available from Agilent Technologies, shown in green) is added at 1 μL/mL. A broad spectrum MMP inhibitor (such as GM6001, available from Selleck Chemicals) is used to evaluate the role of matrix metalloproteinases (MMPs). An exemplary diagram is shown in FIG. 4.

Certain terms are used throughout the description and claims to refer to particular features or components. As one skilled in the art will appreciate, different persons may refer to the same feature or component by different names. This document does not intend to distinguish between components or features that differ in name but not function. Moreover, although various examples given herein recite specific analysis systems, reagents, media, therapeutic agents, cell lines, and the like for purposes of illustrating the underlying inventive concepts in practical terms, the present disclosure contemplates that persona of ordinary skill in the relevant art will be able to substitute, without undue experimentation, various different elements appropriate for their purposes when conducting an assay for determining the effectiveness or interactions between prospective therapeutic agents and various cells lines using different imaging and sensing systems, sizes/makes of well plates, software to control relevant devices, etc.

As used herein, the term “optimize” and variations thereof, is used in a sense understood by data scientists to refer to actions taken for continual improvement of a system relative to a goal. An optimized value will be understood to represent “near-best” value for a given reward framework, which may oscillate around a local maximum or a global maximum for a “best” value or set of values, which may change as the goal changes or as input conditions change. Accordingly, an optimal solution for a first goal at a given time may be suboptimal for a second goal at that time or suboptimal for the first goal at a later time.

As used herein, various chemical compounds are referred to by associated element abbreviations set by the International Union of Pure and Applied Chemistry (IUPAC), which one of ordinary skill in the relevant art will be familiar with. Similarly, various units of measure may be used herein, which are referred to by associated short forms as set by the International System of Units (SI), which one of ordinary skill in the relevant art will be familiar with.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

### ENUMERATED EMBODIMENTS

1. A method of assessing cytolysis of cancer cells by effector cells, the method comprising:
providing a cell-substrate impedance monitoring device operably connected to an impedance analyzer, wherein the device comprises a well for receiving cells and an electrode array at a base of the well, the device further operably connected to an imaging unit;
adding target cells characterized as cancer cells to the well;
disposing a layer comprising an extracellular matrix (ECM) over the target cells;
adding effector cells over the ECM layer; and
imaging the well and monitoring cell-substrate impedance of the well to determine invasion of the effector cells through the ECM layer and effectiveness of effector cell killing of the target cells either directly or via migration and invasion through extracellular matrix.

2. The method of embodiment 1, further comprising an imaging device disposed adjacent to the well.

3. The method of embodiment 1 or 2, the cancer cells are from a solid tumor or a metastatic lesion thereof, optionally wherein the solid tumor is a carcinoma, or a cancer of prostate, breast, lung and bronchus, colon and rectal, urinary bladder, thyroid, kidney and renal pelvis, uterine corpus, oral cavity, or ovarian.

4. The method of any of embodiments 1-3, wherein the effector cells comprise an immune cell (e.g., an immune cell described herein), e.g., a natural killer (NK) cell, a T cell (e.g., CD8+ T cell, cytotoxic T cell), a macrophage, or a combination thereof.

5. The method of any of embodiments 1-3, wherein the effector cells comprise an NK cell.

6. The method of any of embodiments 1-3, wherein the effector cells comprise a T cell.

7. The method of any of embodiments 1-3, wherein the effector cells comprise immune cells, e.g., CAR-T cells, CAR-NK cells, CAR-Macrophages, gamma-delta T cells (e.g., CAR-gd-T cells), or a combination thereof.

8. The method of any of embodiments 1-7, wherein the effector cells are obtained from a subject.

9. The method of embodiment 8, wherein the subject having a cancer.

10. The method of embodiment 9, wherein the cancer is a solid tumor.

11. A method, comprising:
measuring cell-substrate impedances between target cells and effector cells separated by layer of an extracellular matrix (ECM) at various times during an assay;
capturing images the effector cells and the target cells at various times during the assay; and
determining an effectiveness of the effector cells relative to the target cells based on changes to the cell-substrate impedance the images over a time period of the assay.

12. The method of embodiment 11, further comprising:
applying a first dye of a first color to the target cells; and
applying a second dye of a second color, different than the first color, to the effector cells.

13. The method of embodiment 11 or 12, further comprising varying a thickness of the ECM in multiple wells of a multiwell plate in which the target cells and effector cells are disposed.

14. The method of any of embodiments 11-13, further comprising:
applying a pharmaceutical compound of interest to one or more of the target cells, the ECM, and the effector cells;
comparing a baseline effectiveness of the effectiveness the effector cells relative to the target cells without the pharmaceutical compound of interest applied versus an experimental effectiveness of the effectiveness the effector cells relative to the target cells with the pharmaceutical compound of interest applied; and
in response to the experimental effectiveness satisfying an effectiveness threshold, using the pharmaceutical compound of interest for treatment or prophylaxis in a biological subject of a disorder associated with the target cells.

15. The method of any of embodiments 11-13, further comprising:
applying a first pharmaceutical compound of interest to a first subset of one or more of the target cells, the ECM, and the effector cells;
applying a second pharmaceutical compound of interest to a second subset, separate from the first subset, of one or more of the target cells, the ECM, and the effector cells;
comparing a first effectiveness of the effectiveness the effector cells relative to the target cells with the first pharmaceutical compound of interest applied versus a second effectiveness of the effectiveness the effector cells relative to the target cells with the second pharmaceutical compound of interest applied; and
in response to the first pharmaceutical compound of interest having a greater effectiveness than the second pharmaceutical compound of interest, using the first pharmaceutical compound of interest for treatment or prophylaxis in a biological subject of a disorder associated with the target cells.

16. The method of any of embodiments 11-15, wherein the target cells are cancer cells.

17. The method of embodiment 16, wherein the cancer cells are from a solid tumor or a metastatic lesion thereof, optionally wherein the solid tumor is a carcinoma, or a cancer of prostate, breast, lung and bronchus, colon and rectal, urinary bladder, thyroid, kidney and renal pelvis, uterine corpus, oral cavity, or ovarian.

18. The method of any of embodiments 11-17, wherein the effector cells comprise an immune cell (e.g., an immune cell described herein), e.g., a natural killer (NK) cell, a T cell (e.g., CD8+ T cell, cytotoxic T cell), a macrophage, or a combination thereof.

19. The method of any of embodiments 11-17, wherein the effector cells comprise an NK cell.

20. The method of any of embodiments 11-17, wherein the effector cells comprise a T cell.

21. The method of any of embodiments 11-17, wherein the effector cells comprise immune cells, e.g., CAR-T cells, CAR-NK cells, CAR-Macrophages, gamma-delta T cells (e.g., CAR-gd-T cells), or a combination thereof.

22. The method of any of embodiments 11-21, wherein the effector cells are obtained from a subject.

23. The method of embodiment 22, wherein the subject having a cancer.

24. The method of embodiment 23, wherein the cancer is a solid tumor.

25. A method of treating a solid tumor having, or being identified as having, aberrant FGFR signaling, comprising administering a therapeutically effective amount of an FGFR inhibitor (e.g., an inhibitor of FGFR1, FGFR2, FGFR3, FGFR4, or any combination thereof) and a CAR therapy to a subject in need thereof.

26. The method of embodiment 25, wherein the FGFR inhibitor comprises pemigatinib.

27. The method of embodiment 25 or 26, wherein the FGFR inhibitor is administered prior to the CAR therapy is administered.

28. The method of any of embodiments 25-27, wherein the FGFR inhibitor is administered concurrently with the administration of the CAR therapy.

29. The method of any of embodiments 25-28, wherein the FGFR inhibitor is administered after the CAR therapy is administered.

30. The method of any of embodiments 25-29, wherein the CAR therapy comprises immune cells expressing a chimeric antigen receptor comprising an antigen recognition domain, a hinge region, a transmembrane domain, and an intracellular cell signaling domain.

31. The method of embodiment 30, wherein the antigen recognition domain binds to a tumor antigen, optionally wherein the solid tumor is a carcinoma, or a cancer of prostate, breast, lung and bronchus, colon and rectal, urinary bladder, thyroid, kidney and renal pelvis, uterine corpus, oral cavity, or ovarian.

32. The method of any of embodiments 25-31, wherein the CAR therapy is a CAR-T therapy.

33. An FGFR inhibitor (e.g., an inhibitor of FGFR1, FGFR2, FGFR3, FGFR4, or any combination thereof) for use in a method of treating a solid tumor having, or being identified as having, aberrant FGFR signaling in a subject in combination with a CAR therapy.

34. The FGFR inhibitor for use of embodiment 33, wherein the FGFR inhibitor comprises pemigatinib.

35. The FGFR inhibitor for use of embodiment 33 or 34, wherein the FGFR inhibitor is administered prior to the CAR therapy is administered.

36. The FGFR inhibitor for use of any of embodiments 33-35, wherein the FGFR inhibitor is administered concurrently with the administration of the CAR therapy.

37. The FGFR inhibitor for use of any of embodiments 33-36, wherein the FGFR inhibitor is administered after the CAR therapy is administered.

38. The FGFR inhibitor for use of any of embodiments 33-37, wherein the CAR therapy comprises immune cells expressing a chimeric antigen receptor comprising an antigen recognition domain, a hinge region, a transmembrane domain, and an intracellular cell signaling domain.

39. The FGFR inhibitor for use of embodiment 38, wherein the antigen recognition domain binds to a tumor antigen, optionally wherein the solid tumor is a carcinoma, or a cancer of prostate, breast, lung and bronchus, colon and rectal, urinary bladder, thyroid, kidney and renal pelvis, uterine corpus, oral cavity, or ovarian.

40. The FGFR inhibitor for use of any of embodiments 33-39, wherein the CAR therapy is a CAR-T therapy, a CAR-NK cell therapy, a CAR-macrophage therapy, or a CAR-gd-T therapy.

### EXAMPLES

### Example 1

### Evaluating the Invasion and Cytotoxicity of Immune Cells Using Impedance and Imaging

It was hypothesized that matrigel layer presents a challenge for the NK-92 cells and increasing the distance for invasion would delay the killing of tumor cells. Furthermore, the invasion/migration through matrigel is suggested to depend on matrix-metalloproteinases (MMP) that cleave the components of ECM. Therefore, the effects of inhibiting MMP function were also evaluated.

The results demonstrate that the methods and systems described herein can be easily used to systematically study the effects of ECM constituents on tumor and immune cell interactions in the TME according to user requirements.

### Materials and Methods

### Cells

MCF-7 human breast adenocarcinoma cell line (available from ATCC, Cat#HTB-22) were transduced with eLenti Red (available from Agilent Technologies, cat.#8711011) at a multiplicity of infection of one and cultured in the presence of puromycin (2 micrgrams (μg)/mL) for fourteen days to select for MCF-7-red cells stably expressing nuclear-localized red fluorescent protein (RFP). Both MCF-7 and MCF-7-red cells were maintained in culture in Eagle's Mimimum Essential Medium (EMEM, available from ATCC, 30-2003) supplemented with 10% heat inactivated fetal bovine serum (FBS) (available from Sigma, cat.#12106C-500 ML) and 1% Pen/Strep (available from Hyclone, cat.#SV30010).

NK-92 cells (available from Creative Bioarray, CSC-C0499 and ATCC) were grown in MyeloCult H5100 media (available from Stemcell Technologies, Cat.#05150) supplemented with 30 mL horse serum (available from Gibco, Cat#16050-122), 600 IU/mL of rhIL-2 (available from Stemcell Technologies, cat.#78036) and 1% Pen/Strep (available from Hyclone, cat. #SV 30010). All cell lines were maintained at 37 degrees Celsius (C) with 5% Carbon Dioxide (CO₂).

ECM Invasion and Cytotoxicity Assay

The ability of NK-92 cells to invade the matrigel and kill the MCF-7-red target cells was evaluated by concurrent impedance and imaging readouts on an XCELLIGENCE RTCA ESIGHT® imaging and sensing system (available from Agilent Tech., Inc.). Background impedance signal was measured with 50 μL of EMEM media in the wells of E-plate VIEW microplate (available from Agilent Technologies, Cat#0030060101030). MCF-7-red target cells (30,000 in 100 μL) were added to each well. The plate was placed at room temperature for thiry minutes to facilitate an even distribution of cells at the bottom. The plate was then transferred to the XCELLIGENCE RTCA ESIGHT® imaging and sensing system, and data acquisition was initiated. Impedance readings were collected every fifteen minutes, and photos were taken every sixty minutes. Images from four fields of view were acquired in each well in brightfield, red, and green fluorescence channels. Exposure time was set at default in bright field and 150 milliseconds (ms) in the red and green channels. Matrigel (available from Corning, Cat#356234, 354234) was thawed overnight at 4 degrees C, diluted with Dulbecco's Modified Eagle Medium (DMEM) and supplemented with 10% FBS to a final total protein concentration of 6 mg/mL. After twenty-fout hours, data collection was paused. Media in the wells were aspirated and varying volumes of matrigel (e.g., 50, 75 and 100 μL, data shown here) were layered over the MCF-7-red cells. eLive Green (available from Agilent Technologies, Cat#8711003) was added to the matrigel at a final concentration of 1:1000. Wells without ECM were replenished with EMEM (100 μL) containing eLive Green. The plate was incubated for one hour at 37 degrees C at 5% CO₂ for solidification of the matrigel. Impedance and imaging data were collected for an hour during which the NK-92 cells were suspended in EMEM media with eLive Green, and the cell numbers were adjusted to achieve E:T of 3:1 in 100 μL. 100 μL of NK-92 cell suspension was layered over the matrigel, and total volume in all wells was adjusted to 200 μL. The plate was loaded back into the cradle of the XCELLIGENCE RTCA ESIGHT® imaging and sensing system, and data acquisition was resumed for seven days. Percent cytolysis was calculated from normalized cell impedance readings at seventy-two hours using the formula: [(1−(treated/untreated)*100].

### MMO Inhibition and Kinetics of NK Invasion

Ilomostat (GM6001, available from Selleck Chemicals, Cat#S7157), a broad spectrum MMP inhibitor, was dissolved in the matrigel and media at final concentration of 2 micromoles (MM) and 10 μM to assess the role of MMP-dependent remodeling of ECM for NK-92 invasion.

### Results

Target Cell Killing is Delayed with Increasing Invasion Distance for NK Cells

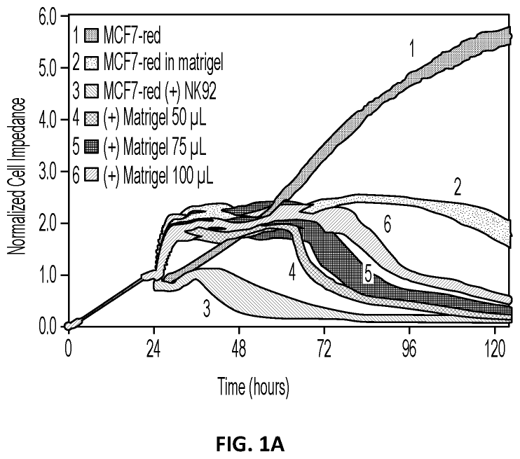

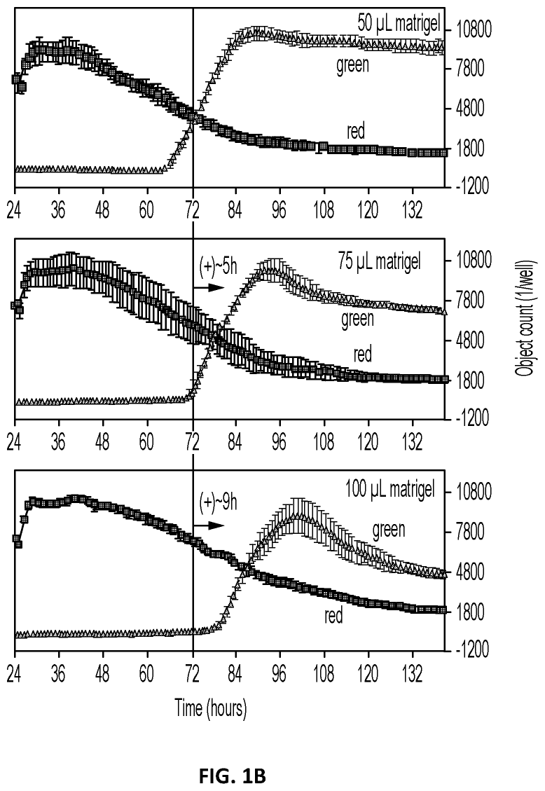

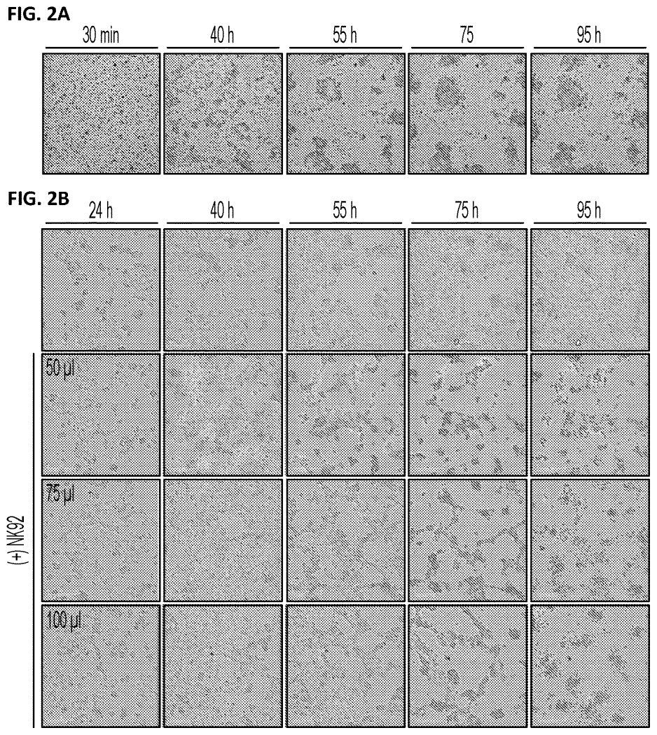

Impedance increases with time as the MCF-7-red target cells adhere and proliferate to stabilize at confluence (FIG. 1A). The addition of NK-92 cells results in a drop in impedance from the loss of adherent target cells during killing. This loss of target cells takes longer in the presence of matrigel, and is delayed with increasing volumes representing greater distance for invasion (FIG. 1A, representative data for 50, 75 and 100 μL).

Image Analysis Corroborates Delayed Invasion and Killing with Increasing Invasion Distance

Images are acquired at the focal plane of tumor cells adhered at the bottom of the wells in the E-view plates (FIG. 1B). In line with the impedance readings, a progressive reduction in red fluorescence associated with the loss of MCF-7-red cells was detected after the addition of NK-92 cells. The green fluorescence increased during the course of the assay in wells with NK-92.Importantly, the kinetics of loss MCF-7(red) cells and gain in green staining (eLive green) after the addition of NKs is delayed with increasing matrigel volume (invasion distance).

### MMP Inhibition Delays Target Cell Killing by NK Cells

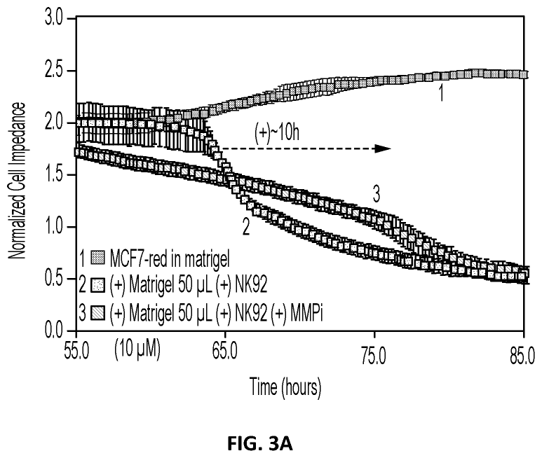

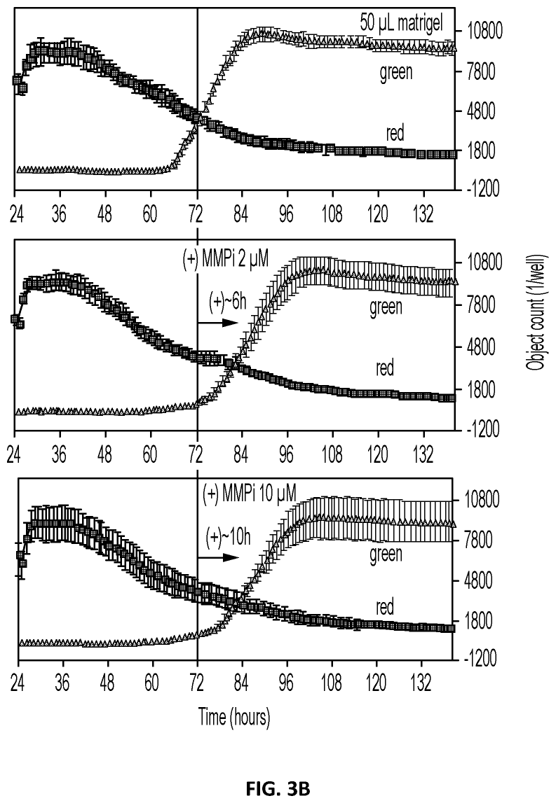

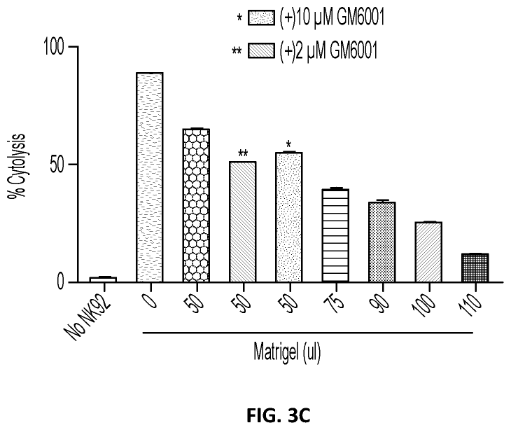

MMPs are suggested to play an important role in lymphocyte invasion. As indicated herein, inhibition of MMPs delays NK-92 cells during invasion, thus slowing down the killing of target cells. Impedance readings (FIG. 3A) revealed that Ilomostat (2 μM and 10 μM) delayed the killing of target cells. Image analysis (FIG. 3B) confirmed the delayed loss of MCF-7(red) and increase in elive Green stained cells with the inhibition of MMP function. The time taken for impedance to fall back to levels at the time of NK-92 addition (normalization time point) increased progressively from 46 h for 50 μL/well to 72 h for 110 μL/well post addition of NKs to the wells. The percent cytolysis at 72 h time point in the assay (FIG. 3C) highlights the delayed killing by the NKs in the presence of increasing invasion distance and presence of the MMP inhibitor.

The drop in impedance readings due to killing of target cells by the invading NK-92 cells slowed down with increasing distance. Consistent with the role of MMPs in NK invasion, the broad spectrum MMP inhibitor slowed down the kinetics of target cell killing in the assay.

Live cell imaging confirmed delayed killing of target cells with increasing invasion distance and in presence of MMP inhibitor. Morphological changes in target cells were detected at ˜45-55 h suggesting distal effects possibly mediated by cytokines.

This assay demonstrates that 96 well format of the XCELLIGENCE RTCA ESIGHT® imaging and sensing system can be adapted to systematically study the interactions of immune and tumor cells in the presence of ECM.

In this Example, the ability of NK-92 cells to invade through varying distances in matrigel to kill tumor cell was evaluated as a proof of concept to establish that this assay format allows simultaneous evaluation of invasion and cytotoxicity in the presence of an ECM. The killing of the target cells is delayed with increasing invasion distance for the NK cells and in the presence of a broad spectrum MMP inhibitor. The cytotoxicity outcome measured by loss in impedance and live cell imaging serves as a surrogate for the migratory/invasion potential of the NK-92 cells. This is relevant for studies focusing on TME and in refining immunotherapy strategies as the ECM can modulate the cellular responses of both invading NK cells and the resident tumor cells. The platform described herein can be used to collect concurrent real-time data in 96 well format for the invasion of extracellular matrix and killing of target cells by lymphocytes.

### Example 2

### Pemigatinib Increases the Effectiveness of EpCAM-CAR-T in Squamous Cell Carcinoma

Chimeric antigen receptor (CAR) T-cell therapy has been generally more effective in hematological malignancies than solid tumors. Considering the complex tumor microenvironment that plays a role in regulating lymphocyte responses and promoting tumor growth, the response rates of CAR-T therapies in solid tumors could be improved by combining with other treatment modalities.

Aberrant fibroblast growth factor (FGF) receptor (FGFR) signaling plays a key role in proliferation and survival of malignant epithelial cells. This study examines the use of a FGF inhibitor in combination with CAR-T cells to target squamous cell carcinoma (SCC) cells in the presence of an extracellular matrix (ECM). The ECM in solid tumors can present a challenge for lymphocyte invasion and act as reservoir of FGF that can contribute to tumor survival and proliferation.

The hypothesis was that inhibition of FGFR signaling would reduce the proliferation of tumor targets resulting in increased killing and improved control of tumor growth by EpCAM-CAR-T cells.

### Materials and Methods

### Cells

A-431 human epidermoid carcinoma cell line (ATCC, Cat#CRL-1555) was transduced with eLenti Red (Agilent Technologies, Cat#8711011) at a multiplicity of infection of 1 and cultured in the presence of 2 μg/mL Puromycin (InvivoGen, Cat#ant-pr-1) for 14 days to select for A431-red cells stably expressing nuclear-localized red fluorescent protein (RFP). A431-red cells were cultured in DMEM media (Corning, 10-013-CV) supplemented with 10% heat inactivated FBS (Sigma, cat#12106C-500 ML) and 1% Pen/Strep (Hyclone, Cat#SV 30010). This media is referred to as c-DMEM in the protocol.

EpCAM-CAR-T cells were cultured in ImmunoCult™-XF T Cell Expansion Medium (Stemcell Technologies, Cat.#10981) supplemented 200 IU/ml of rhIL-2 (Stemcell Technologies, Cat#78036). The T cells were used for the assay on day 4 post-revival.

### Brief Description of Methods

Briefly, matrigel (6 mg/ml) was layered (50 μL/ell) over A431-red target tumor cells expressing nuclear-localized mKate2 (red fluorescent protein). After 2 h, epithelial cell adhesion molecule (EpCAM)-CAR-T cells were seeded over the matrigel layer. Tumor cell growth and CAR-T cytotoxicity were determined from real-time impedance and live cell imaging data collected on the XCELLIGENCE RTCA ESIGHT® imaging and sensing system. Pemigatinib (1 μM, 5 μM and 10 μM), an inhibitor of FGFR isoforms 1-3, sold under the brand name Pemazyre was used to evaluate the role of FGFR signaling. Percent cytolysis was calculated from normalized cell impedance and imaging (red fluorescence) data using the formula [(1−(treated/control)*100].

### ECM Invasion and Cytotoxicity Assay

The ability of EpCAM-CAR-T cells to invade the matrigel and kill A431-red target cells was evaluated using impedance and imaging readouts on an XCELLIGENCE RTCA ESIGHT® imaging and sensing system. The background impedance signal was measured with 50 μL of c-DMEM media in the wells of E-plate VIEW microplate (Agilent Technologies, Cat#0030060101030). Cell suspension of A 431-red cells was prepared in c-DMEM at desired cell numbers to be aliquot at 100 μL/well. After the addition of tumor cells, the E-plate was allowed to rest at room temperature for 30 min to facilitate an even distribution at the bottom. The plate was returned to its cradle in the XCELLIGENCE RTCA ESIGHT® imaging and sensing system to acquire data. Impedance was read every 15 minutes and images were taken every 60 minutes. Images from four fields of view were acquired for each well in brightfield and red fluorescence channel. Exposure time was set at default in bright field and 150 ms in the red channel. Data was collected for 24 hours.

Matrigel (available from Corning, Cat#354234) was thawed overnight at 4° C. and kept on ice while preparing. Matrigel was prepared with Pemigatinib (Selleckchem, Cat#S0088) at 1 μM, 5 μM and 10 μM final concentrations and dimethyl sulfoxide (D2650, Sigma Aldrich) for controls. The matrigel was supplemented with FBS (10% v/v) and total protein concentration was adjusted with DMEM to 6 mg/ml.

Data collection was paused and the plate was taken out of the cradle. The media in the wells were aspirated and matrigel was layered over the A431-red cells. The plate was left to rest at room temperature for 30 minutes and then incubated at 37° C./5% CO₂ for the polymerization of matrigel. Impedance and imaging data were collected for an hour during which the EpCAM-CAR-T cells were suspended in c-DMEM media and the cell numbers were adjusted to achieve desired E:T in 100 μL. The T cell suspension (100 μL) was carefully dispersed over the matrigel. The plate was loaded back into the XCELLIGENCE RTCA ESIGHT® imaging and sensing system cradle and data acquisition was resumed.

### Data Analysis

The software generates a real-time plot of impedance data with time (X-axis) and Cell Index (CI, Y-axis). Cell Index was normalized to the time point before the addition of CAR-T cells. Normalized Cell Index (NCI(t)) was calculated by dividing the CI at time t (CI(t)) with CI at normalization time point (CI(tnormalization)). Thus, the NCI at the normalization time point is set as 1.0 by default. Image analysis was performed at default settings to generate red object counts per well. Percent cytolysis was plotted using the immunotherapy module of software. Percent cytolysis was calculated from NCI (impedance) using the formula [(1−(CAR-T treated/Pemigatinib treated)*100]. Percent cytolysis was calculated from red object counts/well (imaging) using the formula [(1−(CAR-T treated/Pemigatinib treated)*100].

### Results

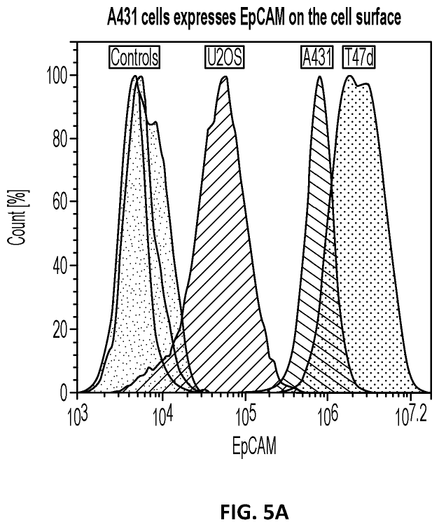

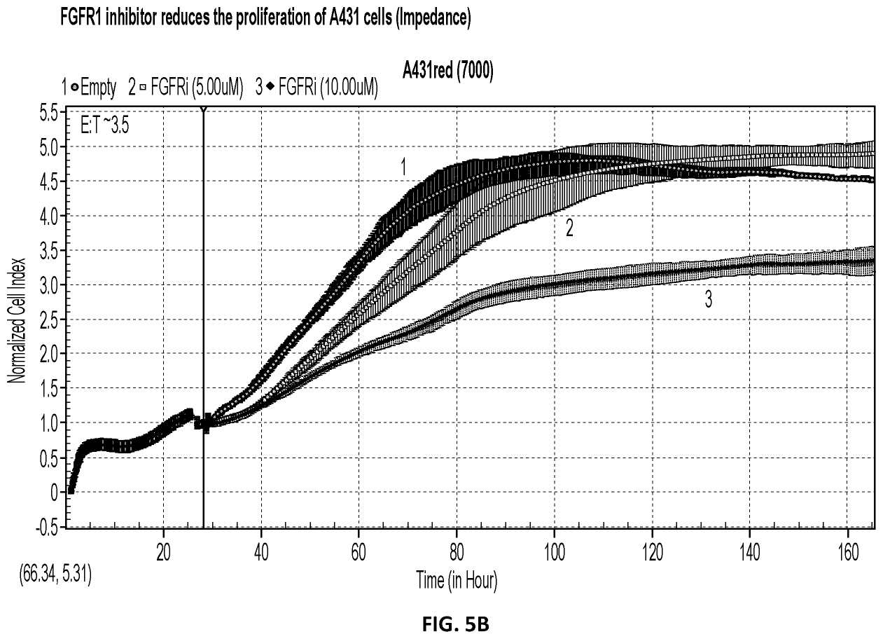

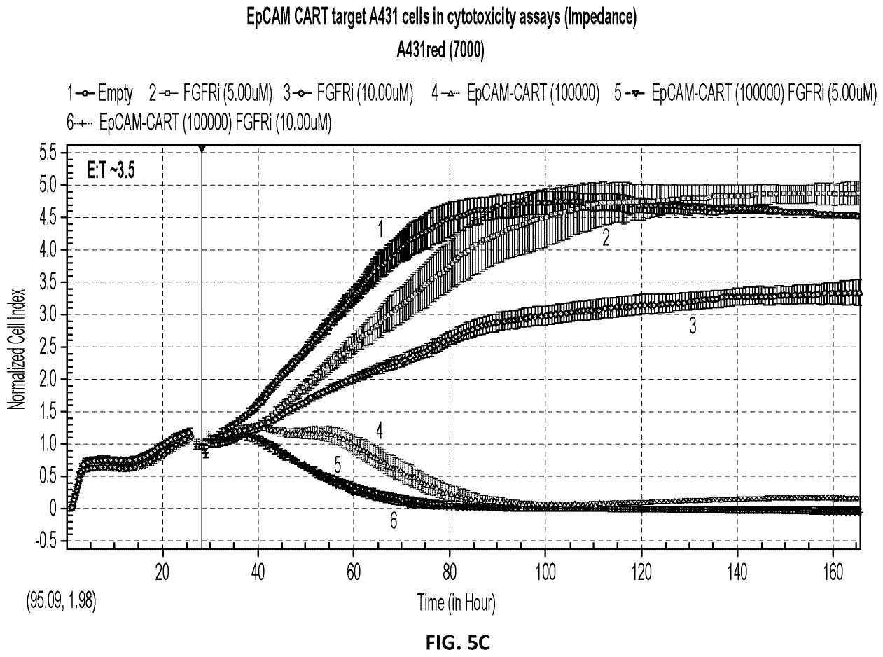

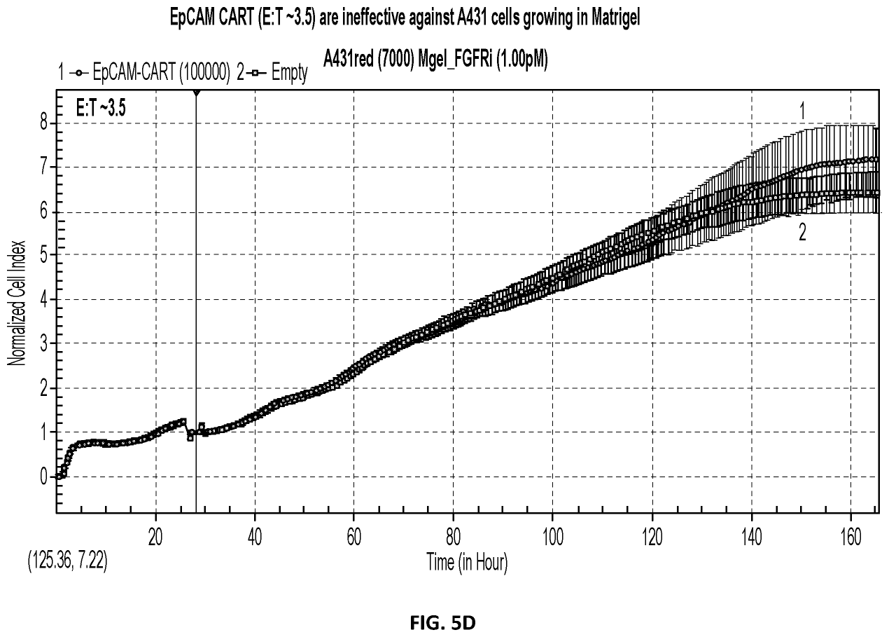

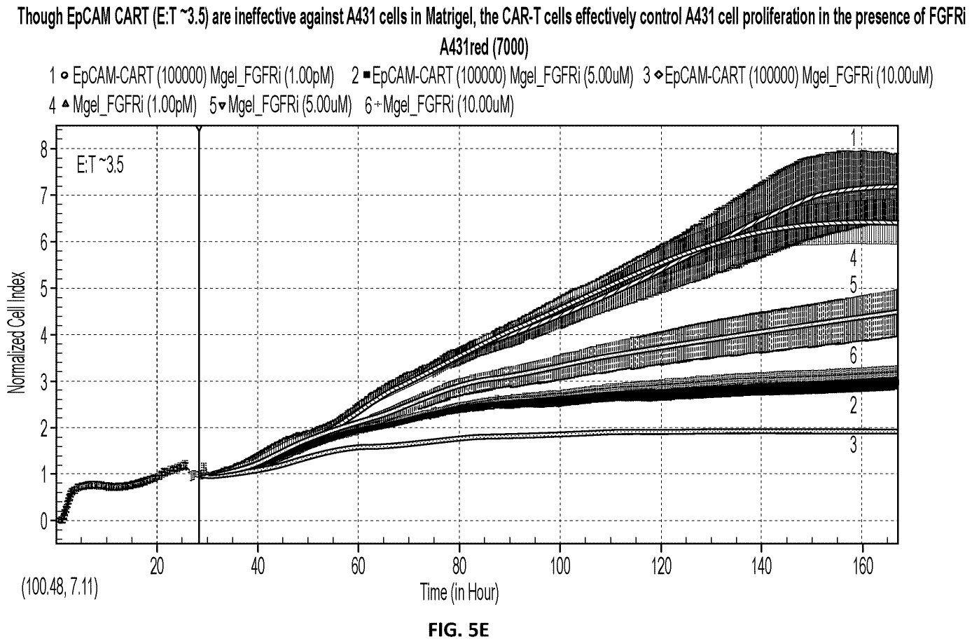

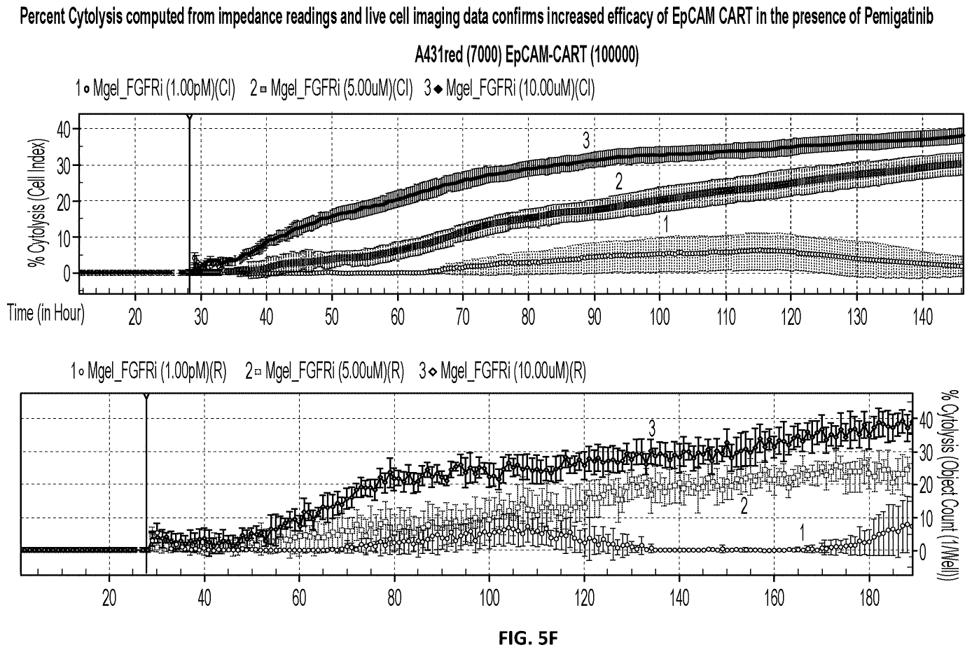

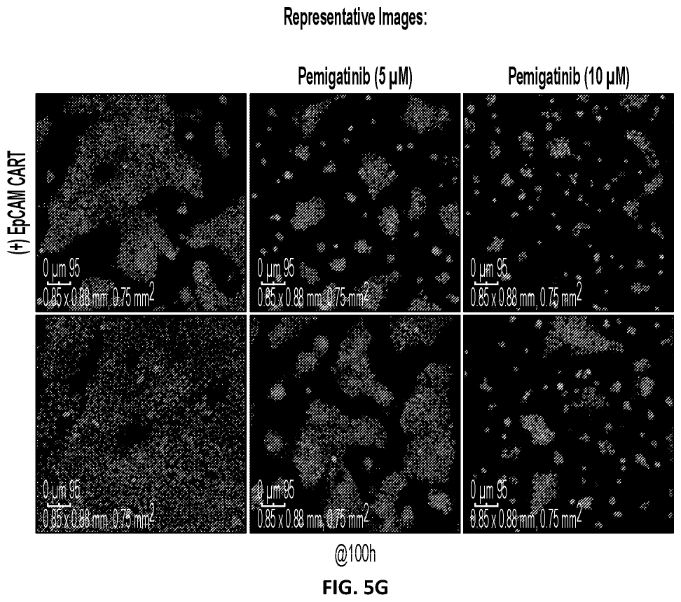

FIGS. 5A-5G illustrate that Pemigatinib increases the effectiveness of EpCAM-CAR-T in squamous cell carcinoma, according to embodiments of the present disclosure, which suggest that Pemigatinib can be effectively combined with CAR-T therapies for solid tumors with aberrant FGFR signaling.

FIG. 5A plots the expression of different cells of EpCAM on the cell surface, where preliminary data from invasion-cytotoxicity assays suggested that EpCAM-CAR-T cells were less effective against A431 cells. Accordingly it is hypothozied that FGFR signaling could play a role in A431 cell proliferation and survival in the presence of matrigel/ECM and that Pemigatinib (Pemazyre, Incyte) inhibits FGFR1-3 isoforms.

FIG. 5B plots the normalized cell index over time shows that FGFR1 inhibitor reduces the proliferation of A431 cells, indicating that Pemigatinib slows the proliferation of A431 cells in native DMEM media in a concentration dependent manner.

FIG. 5C plots EpCAM CART target A431 cells in cytotoxicity assays (FIG. 5C), showing that EpCAM-CAR-T cells trigger the cytolysis of A431 cells (E:T 3.5) cultured in DMEM media. A431 cytolysis is accelerated in the presence of Pemigatinib (5 μM and 10 μM). The data suggest that reduced proliferation aids efficient killing of target cells. E:T is calculated based on the CAR expressing T cells in the transduced T cell population.

FIG. 5D plots that EpCAM CART (E:T ˜3.5) are ineffective against A431 cells growing in Matrigel; EpCAM-CAR-T cells fail to kill A431 cells (E:T 3.5) cultured in matrigel in the absence of Pemigatinib.

FIG. 5E plots the CAR-T cells effectively control A431 cell proliferation in the presence of FGFRi (despite EpCAM CART (E:T ˜3.5) being ineffective against A431 cells in Matrigel), and that Pemigatinib reduces the proliferation of A431 cells in the presence of matrigel in a concentration-dependent manner to improve the outcome of cytolysis by EpCAM-CAR-T cells (E:T 3.5).

FIG. 5F plots that Percent Cytolysis computed from impedance readings and live cell imaging data confirms increased efficacy of EpCAM CART in the presence of Pemigatinib (upper showing percent Cytolysis plotted from normalized Cell Index (NCI) and lower showing normalized red fluorescence count/well). From these results, it is shown that Pemigatinib reduces the proliferation of A431 cells in a concentration-dependent manner to improve the outcome of cytolysis by EpCAM-CAR-T cells (E:T 3.5).

FIG. 5G illustrates representative images taken at the 100 h mark of an assay from which the data plotted in FIGS. 5B-5F were obtained; confirming the hypotheses posited with respect to FIG. 5A.

The example assay and analysis thereof shown in FIGS. 5A-5F was performed using the A431 human epidermoid carcinoma cell line that was transduced with eLenti Red at a multiplicity of infection of 1 and cultured in the presence of 2 μg/mL Puromycin for fourteen days to select for A431-red cells stably expressing nuclear-localized red fluorescent protein (RFP). A431-red cells were cultured in DMEM media supplemented with 10% heat inactivated FBS and 1% Pen/Strep (which may be referred to as c-DMEM).

As shown in assays using the methodologies described herein, a pharmaceutical compound with improved treatment and prophylactic properties has been discovered. These methodologies may be used to identify other pharmaceutical compounds with improved treatment and prophylactic properties for treating various conditions in biological subjects. As demonstrated herein, Pemigatinib can be effectively combined with CAR-T therapies for treating solid tumors with aberrant FGFR signaling. This conclusion is based, at least in part, on the results showing that A431 cells express EpCAM and the EpCAM-CAR-T cells effectively eliminate these tumor cells in cytotoxicity assays. The CAR-Ts were ineffective (Effector:Target, E:T=4:1) in the presence of matrigel. Since FGF signaling can promote the survival and proliferation squamous carcinoma cells, the effect of Pemigatinib, a potent FGFR inhibitor was tested. Pemigatinib reduced the proliferation of A431 cells with increasing concentrations tested. Assessment of CAR-T killing revealed an increase in percent cytolysis from the baseline (˜6%) to ˜20% in the presence of 5 μM Pemigatinib and −40% with 10 μM Pemigatinib. Complete elimination of A 431 cells in invasion assays was achieved with very high E:T=20:1 and 1 μM Pemigatinib.

### Example 3

### Real-Time Co-Culture Assay Using the XCELLIGENCE RTCA ESIGHT Imaging and Sensing System for Immune Cell Invasion and Cytotoxicity

Immune cells extravasate from blood vessels to infiltrate the tissues and perform effector functions that play a crucial role in tumor immune surveillance. These abilities are also harnessed by engineered cytotoxic natural killer (NKs) and CAR (Chimeric Antigen Receptor)-T cell cells used for cancer immunotherapy.

Although cellular immunotherapy has demonstrated effectiveness in hematological cancers, clinical responses in solid tumors need to be improved. The complex tumor microenvironment (TME) in solid tumors regulates lymphocyte recruitment and function. An important challenge faced by lymphocytes in solid tumors is to navigate the acellular space filled with structural scaffolding of the extracellular matrix (ECM) during migration/invasion. This ECM is heterogenous, and its components can modulate a wide array of cellular responses in both resident tumor cells and infiltrating lymphocytes.

Traditionally, invasion, migration and cytotoxicity are evaluated in different endpoint assays using transwell assay systems to predict the in vivo function. The Boyden Chamber is a classical transwell arrangement that is widely used to evaluate migration and invasion, which consists of a cylindrical cell culture insert with a porous membrane nested inside the well of a standard cell culture plate. Cell suspensions are added to the inner chamber of the insert and induced to migrate out through the pores of variable sizes (typically, 3-12 μm) using chemo-attractants in the outer chamber. The inner wells can be coated with ECM to evaluate invasion. The invaded/migrated cells are imaged and/or collected for quantification at predetermined time points. The Boyden Chamber can be modified for evaluating migration and cytotoxicity of effector cells in end-point assays.

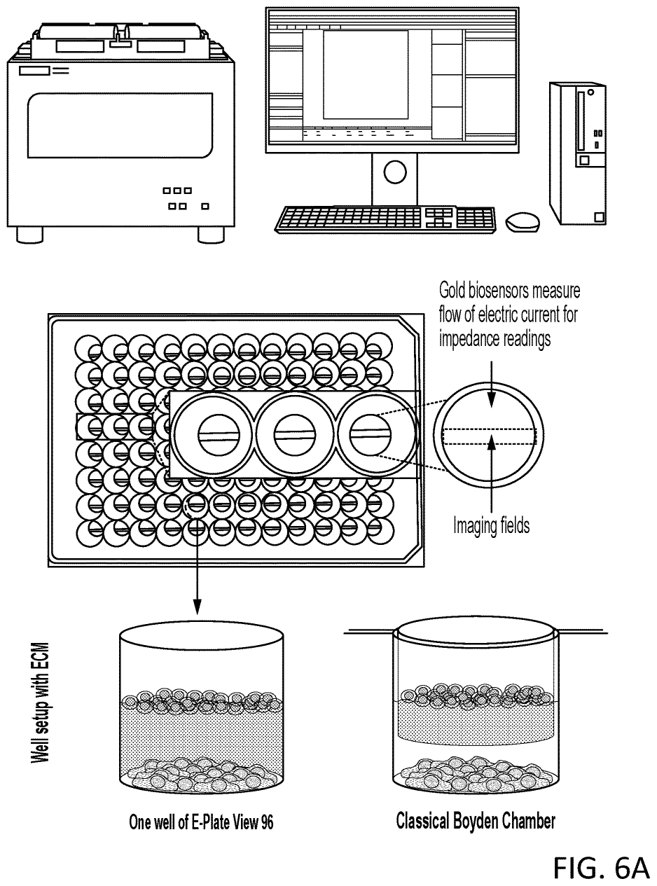

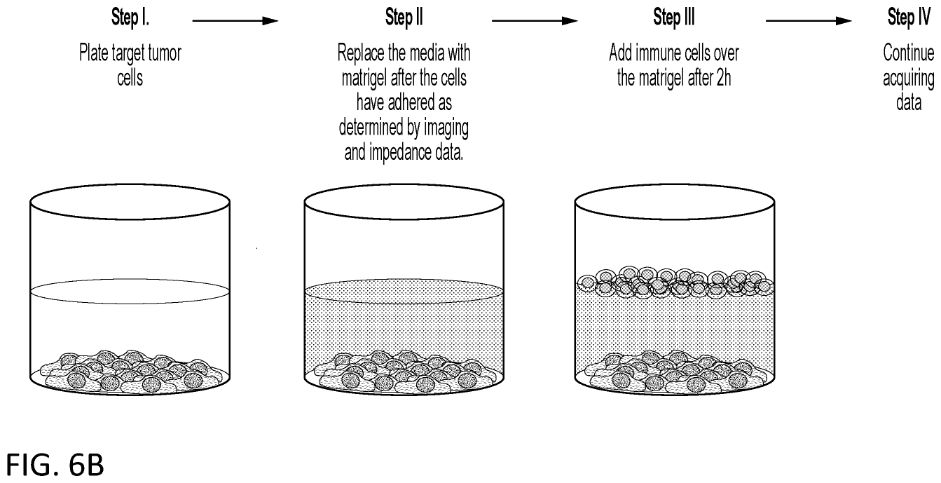

The present disclosure describes a novel real-time co-culture assay using an XCELLIGENCE RTCA ESIGHT® imaging and sensing system for interrogating immune cell invasion and cytotoxicity that does not require the collection of embedded cells for end-point readouts as shown in FIGS. 6A and 6B). In this setup, the ECM layer is in direct contact with target tumor cells and can modulate response of both tumor targets and infiltrating lymphocytes, especially as the latter executes a corresponding cytotoxicity function. In the transwell-based approaches cytotoxicity of the migrated immune cells is carried out in an outer chamber devoid of ECM.

The present example illustrates an evaluation of the invasion and killing of target tumor cells by the NKs as proof of concept for the assay system. NKs are innate lymphocytes that kill infected, stressed or transformed cells by identifying and distinguishing the ‘altered’ cells from healthy cells using an array of activating and inhibiting receptors. The present example uses the NK-92 cells as a model for studying invasion and cytotoxicity. The allogenic NKs derived from NK-92 cell line are the first NK-based cellular immunotherapy to be granted the Investigational New Drug status by the US Food and Drug Administration.

The matrigel layer is hypothesized to present a challenge for the NK-92 cells, and increasing the distance for invasion would delay the killing of tumor cells. Also, NKs rely on proteases of the matrix-metalloproteinases (MMP) family to degrade various components in the ECM during invasion stage.

The present example demonstrate that 96-well format can be easily used systematically study the effects of different ECM constituents on tumor and immune cell interactions in the TME depending on user requirements.

### Materials and Methods

### Cells

The MCF-7 human breast adenocarcinoma cell line was transduced with eLenti Red at a multiplicity of infection of 1 and cultured in the presence of 2 μg/mL Puromycin for fourteen days to select for MCF7-red cells stably expressing nuclear-localized red fluorescent protein (RFP). MCF7 and MCF 7-red cells were cultured in EMEM media supplemented with 10% heat inactivated FBS and 1% Pen/Strep.

NK-92 cells were grown in MyeloCult H5100 media supplemented with 30 ml horse serum, 600 IU/ml of rhIL-2, and 1% Pen/Strep.

eGFP-NK92 cells were grown in X-VIVO 15 supplemented with 5% human serum, 500 IU/ml of rhIL-2, and 0.5 μg/ml Puromycin.

### ECM Invasion and Cytotoxicity Assay

The ability of NK-92 cells to invade the matrigel and kill the MCF7-red target cells was evaluated by concurrent impedance and imaging readouts on an XCELLIGENCE RTCA ESIGHT® imaging and sensing system as outlined in FIG. 6A. Background impedance signal was measured with 50 μL of EMEM media in the wells of E-plate VIEW microplate. MCF 7-red target cells (30,000 in 100 μL) were added to the wells and the plate was placed at room temperature for 30 minutes to facilitate an even distribution at the bottom. The plate was returned to its cradle in the XCELLIGENCE RTCA ESIGHT® imaging and sensing system to acquire data. Impedance was read every fifteen minutes and images were taken every sixty minutes. Images from four fields of view were acquired in each well in brightfield, red, and green fluorescence channels. Exposure time was set at default in bright field, 150 ms in red, and 300 ms in green channels respectively. Matrigel was thawed overnight at 4 degrees C, diluted with DMEM and supplemented with 10% FBS to a final total protein concentration of 6 mg/ml. After twenty-four hours, the data collection was paused. Media in the wells were aspirated and varying volumes of matrigel (50, 75, and 100 μL, data shown here) was layered over the MCF7-red cells and incubated for thirty minutes at room temperature, followed by thirty minutes at 37 degree C/5% CO₂ for polymerization of the matrigel. Impedance and imaging data were collected for an hour, during which the NK-92 cells were suspended in EMEM media and the cell numbers were adjusted to achieve E:T of 3:1 in 100 μL. NK-92 cell suspension (100 μL) was layered over the matrigel and total volume in all wells was adjusted to 200 μL. The plate was loaded back into the cradle, and data acquisition was resumed. Percent cytolysis was calculated from normalized cell impedance readings using the formula [(1−(treated/untreated)*100].

### MMP Inhibition and Kinetics of NK Invasion

Ilomostat, which is a broad spectrum MMP inhibitor, was dissolved in the matrigel and media at a final concentration of 2 μM and 10 μM to assess the role of MMP-dependent NK-92 invasion. Percent cytolysis was calculated from normalized cell impedance readings and red object count data using the formula [(1−(NK92+MMPi/MMPi treated)*100].

### Results

Target Cell Killing is Delayed with Increasing Invasion Distance for NK Cells

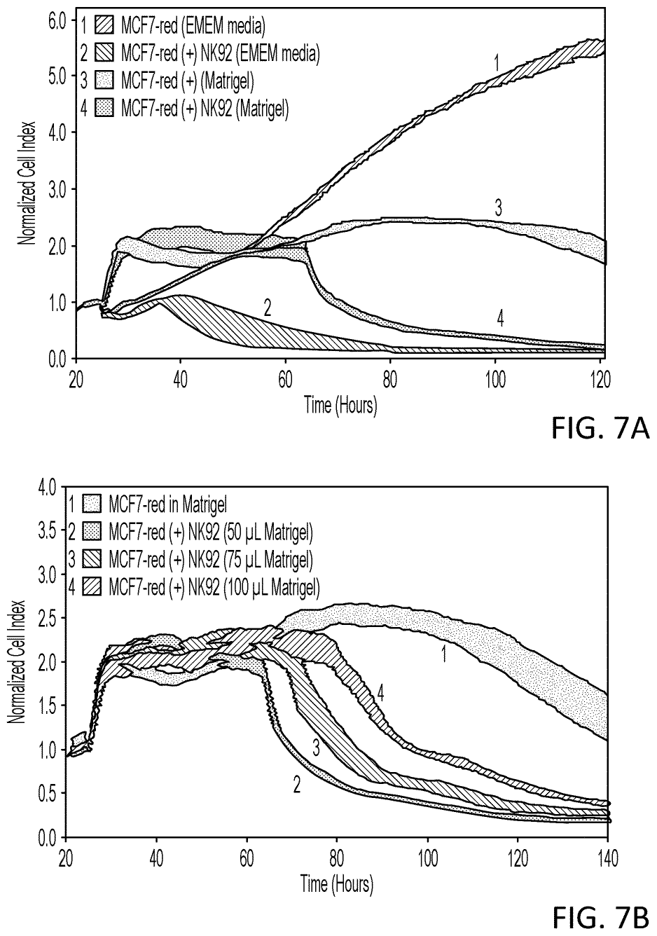

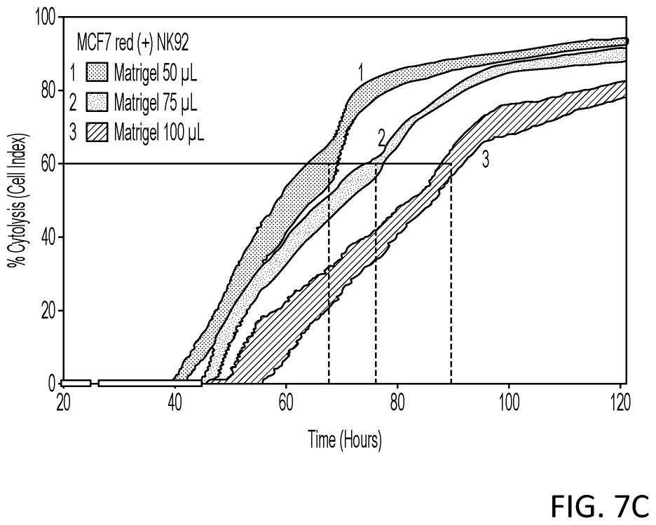

Impedance increases with time and stabilizes as the MCF7-red target cells adhere and proliferate to reach confluence as shown in FIG. 7A. The addition of matrigel (50 μL) modulates the impedance signature of MCF-7 cells and importantly, delays the cytolysis by NK-92 cells added at twenty-four hours. The drop in impedance due to cytolysis is further delayed with increasing volumes of matrigel as shown in FIG. 7B, showing representative data for 50, 75, and 100 μL). The KT60 (time to achieve 60% killing with respect to controls) as shown in FIG. 7C was 67, 76, and 89 hours respectively.

### MMP Inhibition Delays Target Cell Killing by NK Cells

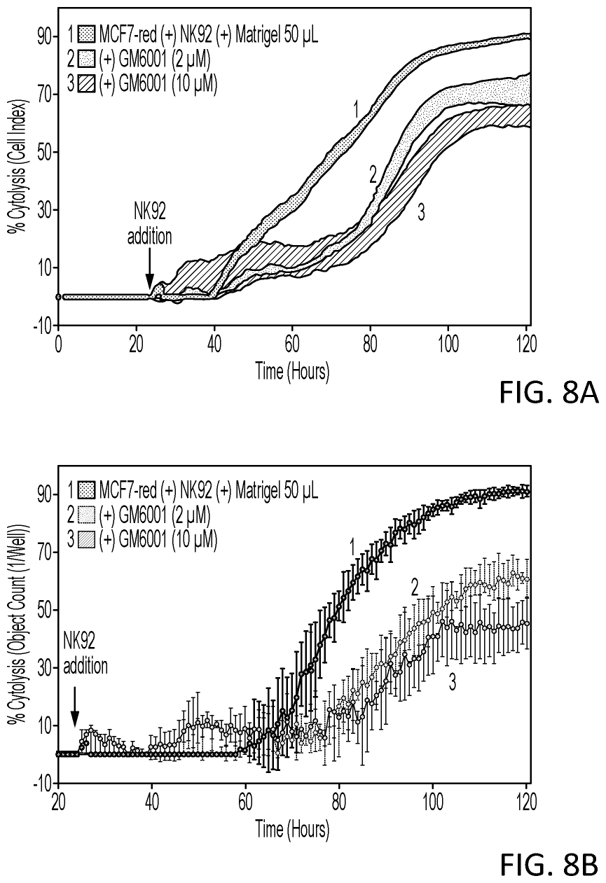

As cytolysis was delayed with increasing invasion distance for the NKs, MMPs are hypothezied to play an important role in lymphocyte invasion. Consistent with the function of MMPs in invasion, percent cytolysis computed from normalized impedance readings as shown in FIG. 8A and live cell imaging (e.g., red fluorescence), as shown in FIG. 8B confirmed both delayed and reduced target cell killing by GFP-NK92 cells in the presence of Ilomostat (2 μM and 10 μM). Image analysis concurs with impedance measurements confirming the kinetics of cell killing.

Image Analysis Corroborates Delayed Invasion and Killing with Increasing Invasion Distance

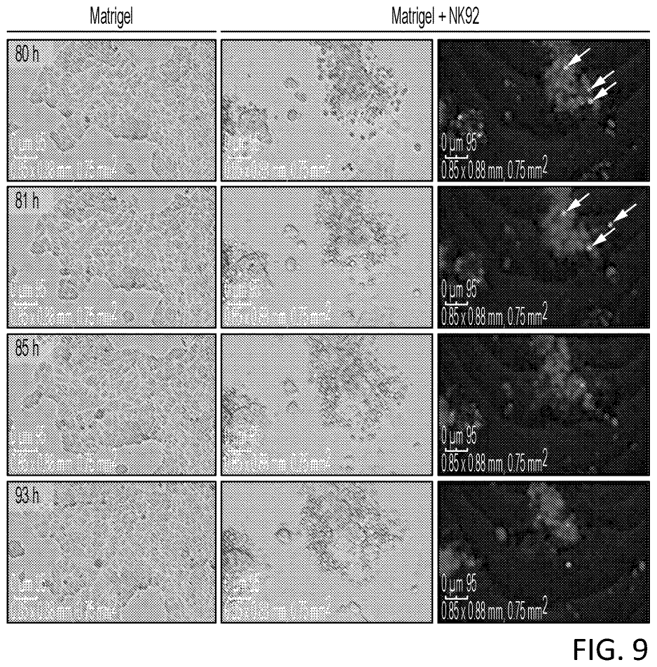

Representative images of MCF-7 clusters, as shown in FIG. 9, reveal increased clumping and cell death in response to GFP-NK92. Although there is increased tumor cell death, only a few GFP-NK92 were detected in the imaging fields. Interestingly, highly active NK cells were detected that made multiple contacts with different MCF-7 red targets in clusters suggesting serial killing activity.

Recapitulating the features of tumor microenvironment in solid tumors could play a pivotal part in formulating and refining the strategies for cellular immunotherapy. The dysregulation of ECM in tumor microenvironment of solid tumors has been widely investigated for its ability to modulate diverse cellular responses. In this regard, there is a need for efficient in vitro assays that evaluate the responses of tumor and immune cells in the presence of ECM.

The ability of NKs and other immune cells to invade the extracellular space to reach the target cells could depend on the ability to degrade ECM. Various constituents of ECM can regulate the function of NK cells. Accordingly, the ability of NK-92 cells to invade a matrigel layer and kill the target cells as described herein bears evaluation. The cytotoxicity outcome measured by loss in impedance and live cell imaging serves as a surrogate for the migratory/invasion potential of the NK-92 cells. This assay format allows simultaneous evaluation of invasion and cytotoxicity in the presence of an ECM by demonstrating that the killing of target cells can be delayed by varying the invasion distance through the matrigel.

NKs can express multiple MMPs and transmigration assays have shown that NK-92 invasion in matrigel is reduced in the presence of MMP inhibitor GM6001. Typically, studies evaluate invasion/migration potential and cytotoxicity functions separately in complex workflows as is the case with the study that evaluated the effect of MMP inhibitor. Alternatively, some studies have evaluated both migration and cytotoxicity using complex cumbersome experimental setups. Live cell imaging and real-time impedance readouts, as described herein, demonstrate the contribution of MMPs in invasion-cytotoxicity assays with NK-92. Consistent with the role of MMPs in facilitating invasion, the killing of target cells is delayed by the broad spectrum MMP inhibitor.

Although imaging and impedance data were consistent in the invasion-cytotoxicty assay, relying only on imaging data can be challenging. Tumor cells respond differently to the matrigel and, as highlighted in the representative images, the MCF-7 target cells form irregular and messy clumps as they undergo cell death. Surprisingly, rather than finding a swarm of invading NK cells, only a few NK cells were detected in and around the MCF clusters. Nonetheless, the images and videos collected per these assays indicate highly active GFP-NK92 cells that made multiple contacts with different targets in the clusters leading to cell death of the contacted targets during the course of the assay.

The novel real-time co-culture assay described in this disclosure demonstrates the utility of the XCELLIGENCE RTCA ESIGHT® imaging and sensing system for the simultaneous evaluation of invasion and cytotoxicity functions of lymphocytes that are critical for immunotherapy in solid tumors.

1.-20. (canceled)

21. A method of assessing cytolysis of cancer cells by effector cells, the method comprising:
providing a cell-substrate impedance monitoring device operably connected to an impedance analyzer, wherein the device comprises a well for receiving cells and an electrode array at a base of the well, the device further operably connected to an imaging unit;
adding target cells characterized as cancer cells to the well;
disposing a layer comprising an extracellular matrix (ECM) over the target cells;
adding effector cells over the ECM layer; and
imaging the well and monitoring cell-substrate impedance of the well to determine invasion of the effector cells through the ECM layer and effectiveness of effector cell killing of the target cells either directly or via migration and invasion through the ECM.

22. The method of claim 21, wherein the ECM comprises a solidified layer of 50-110 microliters in volume that separates the target cells from the effector cells at a time of adding the effector cells over the ECM layer.

23. The method of claim 21, the cancer cell is from a solid tumor of a cancer of prostate, breast, lung and bronchus, colon and rectal, urinary bladder, thyroid, kidney and renal pelvis, uterine corpus, or oral cavity, and wherein the effector cell is a natural killer (NK) cell or a T cell.

24. The method of claim 21, wherein determining the invasion of the effector cells through the ECM layer is based on changes to the cell-substrate impedance data and imaging data over a time period of the assay that is based on a simultaneous acquisition of the cell-substrate impedance data with a cell count from the imaging data.

25. The method of claim 21, further comprising:
applying a first pharmaceutical compound of interest to a first subset of one or more of the target cells, the ECM, and the effector cells;
applying a second pharmaceutical compound of interest to a second subset, separate from the first subset, of one or more of the target cells, the ECM, and the effector cells;
comparing a first effectiveness of the effectiveness the effector cells relative to the target cells with the first pharmaceutical compound of interest applied versus a second effectiveness of the effectiveness the effector cells relative to the target cells with the second pharmaceutical compound of interest applied; and
in response to the first pharmaceutical compound of interest having a greater effectiveness than the second pharmaceutical compound of interest, using the first pharmaceutical compound of interest for treatment or prophylaxis in a biological subject of a disorder associated with the target cells,
wherein the first pharmaceutical compound of interest is a fibroblast growth factor (FGF) receptor (FGFR) inhibitor, and wherein the disorder associated with the target cells has been identified as including aberrant FGFR signaling.

26. A method, comprising:
measuring cell-substrate impedances between target cells and effector cells separated by a solidified layer of an extracellular matrix (ECM) at various times during an assay;
capturing images the effector cells and the target cells at various times during the assay; and
determining an effectiveness of the effector cells relative to the target cells based on changes to the cell-substrate impedance and the images over a time period of the assay.

27. The method of claim 26, further comprising:
applying a first dye of a first color to the target cells; and
applying a second dye of a second color, different than the first color, to the effector cells.

28. The method of claim 26, further comprising varying a thickness of the ECM in multiple wells of a multiwell plate in which the target cells and effector cells are disposed.

29. The method of claim 26, further comprising:
applying a pharmaceutical compound of interest to one or more of the target cells, the ECM, and the effector cells;
comparing a baseline effectiveness of the effectiveness the effector cells relative to the target cells without the pharmaceutical compound of interest applied versus an experimental effectiveness of the effectiveness the effector cells relative to the target cells with the pharmaceutical compound of interest applied; and
in response to the experimental effectiveness satisfying an effectiveness threshold, using the pharmaceutical compound of interest for treatment or prophylaxis in a biological subject of a disorder associated with the target cells.

30. The method of claim 26, further comprising:
applying a first pharmaceutical compound of interest to a first subset of one or more of the target cells, the ECM, and the effector cells;
applying a second pharmaceutical compound of interest to a second subset, separate from the first subset, of one or more of the target cells, the ECM, and the effector cells;
comparing a first effectiveness of the effectiveness the effector cells relative to the target cells with the first pharmaceutical compound of interest applied versus a second effectiveness of the effectiveness the effector cells relative to the target cells with the second pharmaceutical compound of interest applied; and
in response to the first pharmaceutical compound of interest having a greater effectiveness than the second pharmaceutical compound of interest, using the first pharmaceutical compound of interest for treatment or prophylaxis in a biological subject of a disorder associated with the target cells.

31. The method of claim 30, wherein the first pharmaceutical compound of interest is a fibroblast growth factor (FGF) receptor (FGFR) inhibitor and the disorder has been identified as including aberrant FGFR signaling, further comprising:
administering a therapeutically effective amount of the FGFR inhibitor and a chimeric antigen receptor (CAR) therapy to a subject from whom the target cells were provided.

32. The method of claim 26, wherein the target cell is characterized as a cancer cell.

33. The method of claim 32, the cancer cell is from a solid tumor of a cancer of prostate, breast, lung and bronchus, colon and rectal, urinary bladder, thyroid, kidney and renal pelvis, uterine corpus, or oral cavity.

34. The method of claim 26, wherein determining the effectiveness of the effector cells relative to the target cells based on changes to the cell-substrate impedance and the images over a time period of the assay is based on a simultaneous acquisition of the cell-substrate impedance data with a cell count from the images.

35. A fibroblast growth factor (FGF) receptor (FGFR) inhibitor for use in a method of treating a solid tumor with aberrant FGFR signaling in a subject in combination with a chimeric antigen receptor (CAR) therapy.

36. The FGFR inhibitor of claim 35, wherein the FGFR inhibitor comprises pemigatinib.

37. The FGFR inhibitor of claim 35, wherein the FGFR inhibitor is administered prior to when the CAR therapy is administered.

38. The FGFR inhibitor of claim 35, wherein the FGFR inhibitor is administered concurrently with administration of the CAR therapy.

39. The FGFR inhibitor of claim 35, wherein the FGFR inhibitor is administered after the CAR therapy is administered.

40. The FGFR inhibitor of claim 35, wherein the CAR therapy comprises immune cells expressing a chimeric antigen receptor comprising an antigen recognition domain, a hinge region, a transmembrane domain, and an intracellular cell signaling domain, wherein the antigen recognition domain binds to a tumor antigen, wherein the solid tumor is a carcinoma, or a cancer of prostate, breast, lung and bronchus, colon and rectal, urinary bladder, thyroid, kidney and renal pelvis, uterine corpus, or oral cavity.
